(19) United States
(12) Patent Application Publication
(10) Pub. No.: US 2023/0078674 A1
(43) Pub. Date: Mar. 16, 2023

(54) SYSTEMS AND METHODS FOR CONTAINING AND REMOVING AEROSOL DURING A MEDICAL OR DENTAL PROCEDURE

(71) Applicant: Mindy Joy Benjamini, Merion Station, PA (US)

(72) Inventor: Mindy Joy Benjamini, Merion Station, PA (US)

(21) Appl. No.: 18/058,680

(22) Filed: Nov. 23, 2022

Related U.S. Application Data
(63) Continuation of application No. PCT/US2021/070598, filed on May 24, 2021.
(60) Provisional application No. 63/029,531, filed on May 24, 2020.

(51) Int. Cl. A61C 17/14; A61G 15/10; A61B 46/20; A61C 17/06; A45D 44/02

(52) U.S. Cl. CPC A61C 17/14; A61G 15/10; A61B 46/20; A61C 17/065; A45D 44/02

(57) ABSTRACT

Systems, methods and components thereof for preventing ambient spread of aerosol during a cosmetic, medical or dental procedure are disclosed. The systems and methods utilize a containment unit that includes a rigid or semi-rigid transparent shield and a transparent or translucent flexible bib extending from an outer periphery of the shield. The containment unit covers a portion of a patient while still allowing a professional to view and perform a procedure on that portion of the patient. The systems and methods further utilize an inlet that is usable to facilitate evacuation of air from a confined space created by the containment unit.

### CROSS-REFERENCE TO RELATED APPLICATIONS

This application claims priority to U.S. Provisional Patent Application No. 63/029,531, filed May 24, 2020, entitled, “SYSTEMS AND METHODS FOR CONTAINING AND REMOVING AEROSOL DURING A MEDICAL OR DENTAL PROCEDURE”, the contents of which are incorporated herein by reference in their entirety.

### BACKGROUND OF THE INVENTION

### Field of the Invention

The disclosed concept relates to systems and methods for preventing spread of aerosol during a cosmetic, medical or dental procedure. More particularly, the disclosed concept relates to a containment unit that may be placed over a portion of a patient, e.g., over a patient's head and upper torso, during a procedure, to contain aerosol generated during the procedure. Optionally, an inlet for evacuation of aerosol generated during the procedure may be provided within the containment unit to safely remove the aerosol.

### Description of Related Art

Concern for the spread of airborne pathogens is not new. However, the present COVID-19 pandemic has made the issue of preventing the spread of airborne disease much clearer and more present. According to the U.S. Center for Disease Control (CDC), COVID-19 is a new disease thought to transmit mainly between people who are within about six feet of each other. The routes of transmission are currently believed to be direct contact and through aerosolized airborne particles (e.g., droplets or solid particulate), such as from a person's sneeze or cough. This has resulted in governmental recommendations and in some cases, mandates, to practice “social distancing.” However, as human beings cannot remain apart from each other indefinitely, the manner in which people will need to physically interact so as to reduce the likelihood of disease transmission between them, is a topic on the minds of many. Currently, many people are much more conscious about the need for thorough handwashing and for wearing of facemasks in stores, etc., to prevent disease transmission. However, handwashing and facemasks will not address all transmission risks.

Due to the unique nature of dentistry, most dental procedures generate significant amounts of aerosol from a person's mouth, posing potential risks of infection transmission. Dental procedures that use dental instruments such as handpieces, ultrasonic scalers and air-water syringes, create a spray that may contain contaminated droplets or particulate. During the current pandemic, dental health care personnel should utilize recommended personal protective equipment (PPE), including gowns, gloves, eye protection (e.g., face shield or goggles) and facemasks. Further, to minimize potential exposure to a sick patient, only essential staff should be in the room when performing procedures with potential for aerosol generation. In addition, some dental offices have engineering controls to shield people within the office from exposure to airborne disease such as COVID-19. This may include easily decontaminated physical barriers between patient treatment areas (e.g., curtains), local exhaust ventilation to remove aerosols in a room generated during dental care and directional airflow (e.g., fans or suction) to ventilate a room. However, with all these measures, the fact is that dentistry significantly spreads aerosol, which is potentially contaminated, throughout a dental office. Such spread may reach the room in which a procedure is being done and possibly to nearby rooms as well, where unprotected personnel, patients or visitors may be.

Therefore, Applicant has determined that there is a vital need for a system to trap and evacuate particulate at its source—the vicinity of the mouth of the dental patient. However, Applicant also recognizes that such a need also exists for other types of procedures performed on humans, e.g., medical or cosmetic procedures.

### SUMMARY OF THE INVENTION

Accordingly, in one aspect, the disclosed concept provides a system for preventing spread of aerosol during a cosmetic, medical or dental procedure. The system includes a containment unit configured for positioning over a portion of a patient (e.g., head, neck and a portion of the upper torso) so as to define a confined space around the portion of the patient. The containment unit may include a rigid or semi-rigid transparent shield that is bounded by an outer periphery. The containment unit may further include a transparent or translucent flexible bib. The shield should thus permit a clear line of sight through it while the bib can permit a clear line of sight (if transparent) or at least some level of visual perception (if translucent) through it. The bib extends from the outer periphery. For example, the flexible bib may extend downwardly from the entire length of the outer periphery or optionally from all of the outer periphery minus some or all of the outer periphery running along a back section of the shield. The system may further optionally include an inlet configured to be provided within the confined space. The inlet is configured to facilitate evacuation of air, e.g., suction evacuation, from the confined space.

Optionally, in any embodiment, the system includes a mounting arm that is coupled to the shield to facilitate positioning of the containment unit over the portion of the patient. The mounting arm is movable along at least one plane to adjust positioning of the containment unit.

Optionally, in any embodiment of the system, the containment unit is configured to trap the aerosol within the confined space so as to facilitate directional flow of the aerosol towards the inlet. The inlet is optionally coupled to a lumen connected to a vacuum source to provide suction evacuation of air from the confined space.

Optionally, in any embodiment of the system, the shield is made from a polymer or glass that is treated or coated with an antifogging material, an antimicrobial material and/or an anti-glare material.

Optionally, in any embodiment of the system, the bib extends from a majority of the outer periphery, optionally from at least 75% of the outer periphery, optionally from at least 85% of the outer periphery, optionally from at least 90% of the outer periphery, optionally from the substantial entirety of the outer periphery, optionally from the entirety of the outer periphery.

Optionally, in any embodiment of the system, the inlet is coupled to a lumen connected to a vacuum source to provide suction evacuation of air from the confined space so as to create a negative pressure zone within the confined space.

Optionally, in any embodiment of the system in which the inlet is a funnel, the funnel has an outer periphery, a portion of which is flat.

Optionally, in any embodiment of the system, one or more fasteners removably secure the bib to the shield. Optionally, the one or more fasteners may include one or more of: an adhesive, adhesive tab, putty, clip, hook and eye, nub and eye and hook and loop.

Optionally, in any embodiment of the system, one or more fasteners removably secure the bib to the patient or to a chair supporting the patient. Optionally, the one or more fasteners may include: an adhesive, adhesive tab, putty, clip, hook and eye, nub and eye, hook and loop, straps, ties and static cling.

Optionally, in any embodiment of the system, the system includes one or more filtration elements in the inlet or downstream from the inlet for trapping particulates or decontaminating the air drawn into the inlet from the confined space.

Optionally, in any embodiment of the system, the containment unit is positioned over the patient's entire face and neck and at least a portion of the patient's upper torso, so as to contain aerosol generated from the procedure. Optionally, the inlet is coupled to a lumen connected to a vacuum source to provide suction evacuation of air from the confined space, thereby evacuating the aerosol.

In an optional embodiment, the disclosed concept is directed to a chair for supporting a patient undergoing a cosmetic, medical or dental procedure. The chair is coupled to a portion of a system for preventing spread of aerosol during the procedure. The system to which the chair is coupled includes a containment unit configured for positioning over a portion of the patient so as to define a confined space around the portion of the patient. The containment unit includes a rigid or semi-rigid transparent shield that is bounded by an outer periphery and a transparent or translucent flexible bib extending from the outer periphery. An inlet is configured to be provided within the confined space, wherein the inlet is configured to facilitate evacuation of air from the confined space. A mounting arm is secured to the chair and extends therefrom. The mounting arm is coupled to the shield of the containment unit to facilitate positioning of the containment unit over the chair. The mounting arm is movable along at least one plane to adjust positioning of the containment unit.

Optionally, in any embodiment of the chair, the containment unit is positioned such that at least a portion of the bib extends from the shield in a direction that is not downward relative to the shield and wherein the bib is removably secured to the chair or to a patient supported by the chair with one or more fasteners.

In another aspect, the disclosed concept is a method for preventing spread of aerosol during a cosmetic, medical or dental procedure. The method includes providing a containment unit over a portion of a patient so as to define a confined space around the portion of the patient. The containment unit may include a rigid or semi-rigid transparent shield that is bounded by an outer periphery. The containment unit further may include a transparent or translucent flexible bib extending from at least a substantial length of the outer periphery. The method may further optionally include providing an inlet within the confined space to facilitate evacuation of air, e.g., suction evacuation, from the confined space.

Optionally, in any embodiment of the method, a mounting arm is provided that is coupled to the shield to facilitate positioning of the containment unit over the portion of the patient. The mounting arm is movable along at least one plane to adjust positioning of the containment unit.

Optionally, in any embodiment of the method, the bib extends from a majority of the outer periphery, optionally from at least 75% of the outer periphery, optionally from at least 85% of the outer periphery, optionally from at least 90% of the outer periphery, optionally from the substantial entirety of the outer periphery, optionally from the entirety of the outer periphery.

Optionally, in any embodiment of the method, the inlet is coupled to a lumen connected to a vacuum source to provide suction evacuation of air from the confined space so as to create a negative pressure zone within the confined space.

Optionally, in any embodiment of the method, the containment unit is positioned over the patient's entire face and neck and at least a portion of the patient's upper torso, so as to contain aerosol generated from the procedure which is performed on the patient's mouth.

Optionally, in any embodiment of the method, the containment unit traps the aerosol within the confined space so as to facilitate directional flow of the aerosol towards the inlet. The inlet is coupled to a lumen connected to a vacuum source to provide suction evacuation of air from the confined space, thereby evacuating the aerosol.

Optionally, in any embodiment of the method, the aerosol is generated from the procedure and the site of the patient's body undergoing the procedure is visible from outside of the containment unit to enable a professional who is performing the procedure to see the site so as to perform the procedure within the containment unit. Optionally, the site of the procedure is the patient's mouth or teeth and the professional can see inside of the patient's mouth from outside the containment unit. In such an embodiment, the containment unit is positioned over the patient's entire face and neck and at least a portion of the patient's upper torso, so as to contain aerosol generated from the procedure. Optionally, the containment unit is positioned and configured such that the professional can extend a hand or an instrument into the containment unit by reaching underneath the bib to perform the procedure.

Optionally, in any embodiment of the method, the inlet is a funnel with an outer funnel periphery and the outer funnel periphery has a flat portion.

Optionally, the disclosed concept is directed to the containment unit itself as described herein.

Optionally, the disclosed concept is directed to a kit for preventing spread of aerosol during a cosmetic, medical or dental procedure. The kit includes a rigid or semi-rigid transparent polymer or glass shield that is bounded by an outer periphery. The outer periphery is configured to have a bib secured thereto. The kit further includes at least one transparent or translucent flexible polymer bib configured to be secured to a majority (or substantial entirety or entirety) of the outer periphery so as to form a containment unit configured to be placed over a portion of a patient so as to define a confined space around the portion of the patient. The kit optionally further includes an inlet configured to be placed within the confined space and coupled to a lumen connected to a vacuum source to provide suction evacuation of air from the confined space. The kit optionally further includes a mounting arm configured to be coupled to the shield to facilitate positioning of the containment unit over the portion of the patient. The mounting arm is movable along at least one plane to adjust positioning of the containment unit.

In optional embodiments of the method, system or kit described herein, the inlet is included. In alternative optional embodiments of the method, system or kit described herein, the inlet is not included.

### BRIEF DESCRIPTION OF THE DRAWINGS

The invention will be described in conjunction with the following drawings in which like reference numerals designate like elements and wherein:

FIG. 1 is a top view of certain components of a kit and system according to an optional embodiment of the disclosed concept;

FIG. 2 is an isometric view of an inlet in the form of a funnel usable with a system according to an optional embodiment of the disclosed concept.

FIG. 3A is a side view of a chair comprising components of a system according to an optional embodiment of the disclosed concept.

FIG. 3B is a front partial isometric view of the chair and components of the system of FIG. 3A.

FIG. 3C is another front partial isometric view of the chair and components of the system of FIG. 3A.

FIG. 4A is a top view of a bib according to an optional embodiment of the disclosed concept, when laid out flat.

FIG. 4B is a bottom isometric view of a containment unit comprising a shield and bib according to an optional embodiment of the disclosed concept.

FIG. 4C is a top view of the containment unit of FIG. 4B.

FIG. 5 is a top isometric view of an optional shield of the disclosed concept to which a mounting arm is coupled.

FIG. 5A is an enlarged view illustrating coupling of the mounting arm to the shield utilizing screws as fasteners.

FIG. 6A is a side view of an optional clamp mechanism that is configured to removably secure the mounting arm to a chair or other structure.

FIG. 6B is an isometric view of the clamp mechanism of FIG. 6A with the mounting arm coupled thereto.

FIG. 7A is a side isometric view of an optional embodiment of the system according to the disclosed concept, being used during a dental procedure.

FIG. 7B is a top isometric view of the system of FIG. 7A, as used during the dental procedure.

FIG. 7C is a side isometric view of the system of FIG. 7A, as used during the dental procedure.

FIG. 7D is another top isometric view of the system of FIG. 7A, as used during the dental procedure.

FIG. 7E is an isometric view of the system of FIG. 7A from within the containment unit of the system when the containment unit is positioned over the patient undergoing the dental procedure.

FIG. 8A is a top view of an alternative shield configuration according to an optional aspect of the disclosed concept.

FIG. 8B is a top view of another alternative shield configuration according to an optional aspect of the disclosed concept.

FIG. 8C is an isometric view of another alternative shield configuration according to an optional aspect of the disclosed concept.

FIG. 8D is a front perspective view of another alternative shield configuration according to an optional aspect of the disclosed concept.

FIG. 9A is a top isometric view of the system according to an optional embodiment of the disclosed concept being used with a patient undergoing knee surgery.

FIG. 9B is top isometric view of the system according to an optional embodiment of the disclosed concept being used with a patient undergoing open heart surgery.

FIG. 9C is a top isometric view of the system according to an optional embodiment of the disclosed concept being used with a patent undergoing a tonsillectomy.

FIG. 9D is a side isometric view of the system according to an optional embodiment of the disclosed concept being used with a customer receiving facial cosmetic treatment from a beautician.

### DETAILED DESCRIPTION OF PREFERRED EMBODIMENTS

As used herein, the term “aerosol” is defined as a suspension of fine solid or liquid particles in gas. Aerosols may include, but are not limited to, airborne liquid droplets, mucus droplets, biological airborne contaminants (e.g., viral particles or bacteria contained in or on droplets or particulates) and particles of biological human tissue (e.g., from teeth, gums, skin, bones or internal organs) that may scatter into surrounding air during a cosmetic, medical or dental procedure. Such aerosol may be generated, e.g., by dental instruments, which when used in a patient's mouth, create a spray of aerosol that may contain contaminated droplets or particulate.

As used herein, the terms “rigid” or “semi-rigid,” as applied to a component or material, means that the component or material maintains its shape under gravity. As used herein, the term “flexible,” as applied to a component or material, means that the component or material does not maintain its shape under gravity.

As used herein, the term “confined space” refers to a substantially enclosed volume, that while not air tight, is configured to substantially limit the spread of aerosol generated within the confined space to an area outside of the confined space.

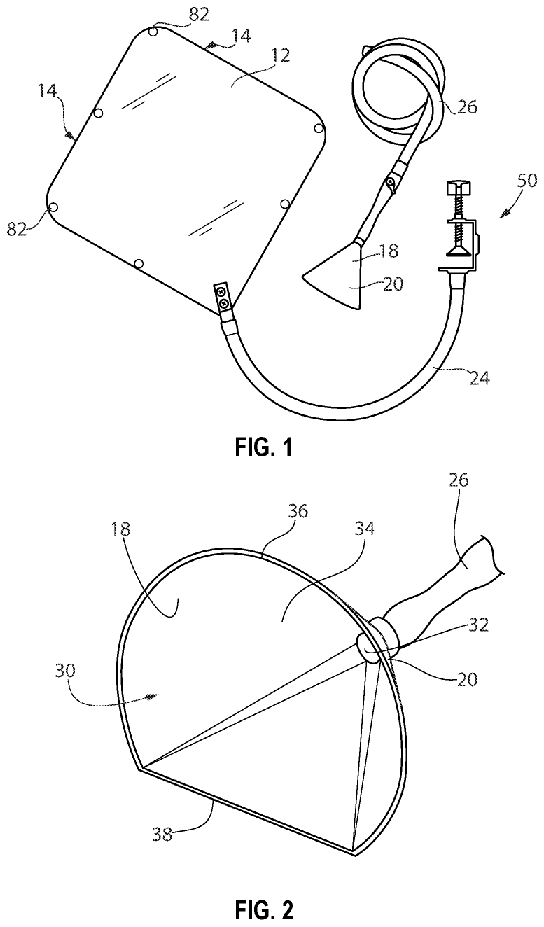

Referring now in detail to the various figures of the drawings wherein like reference numerals refer to like parts, there is shown in FIGS. 1 to 9D optional embodiments of systems according to the disclosed concept, optional uses of such systems and components and subassemblies of such systems.

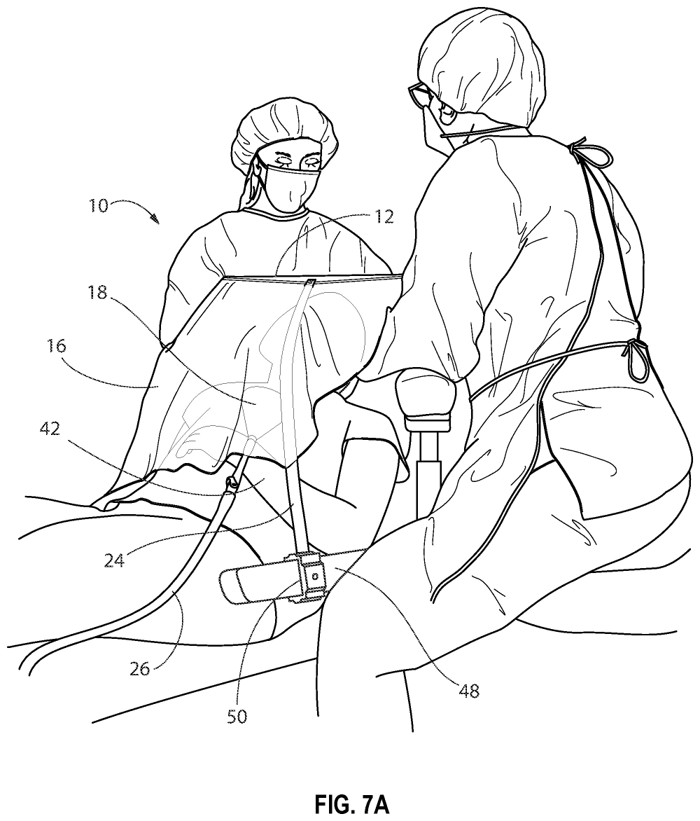

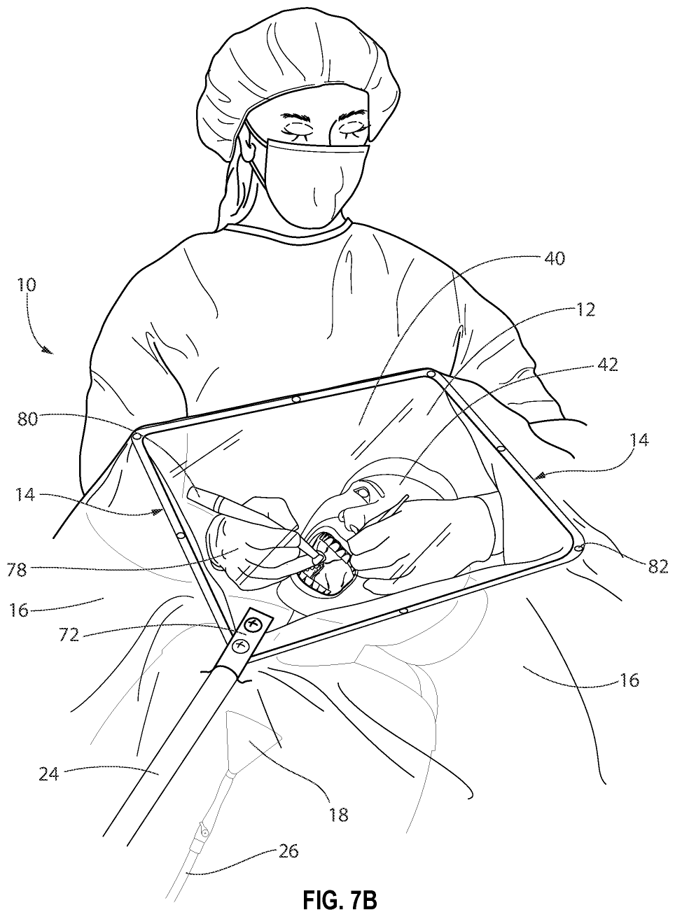

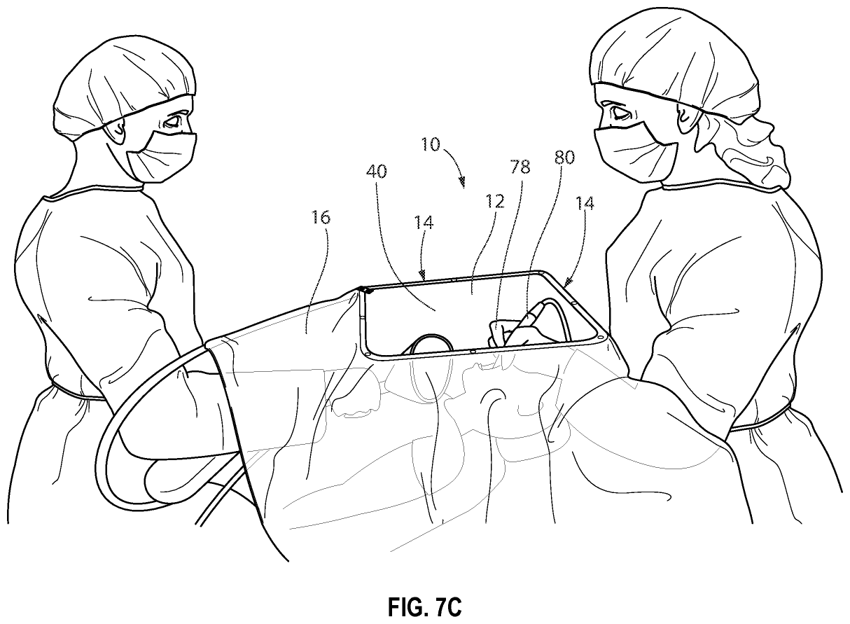

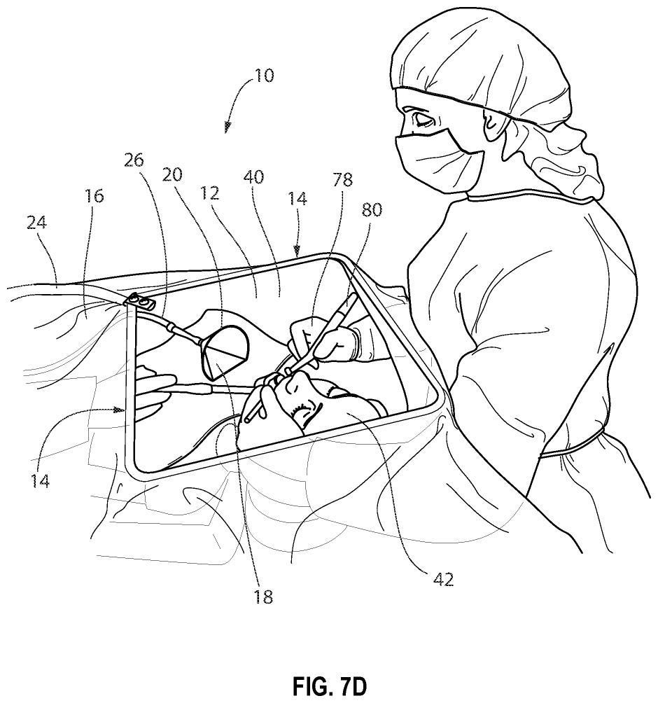

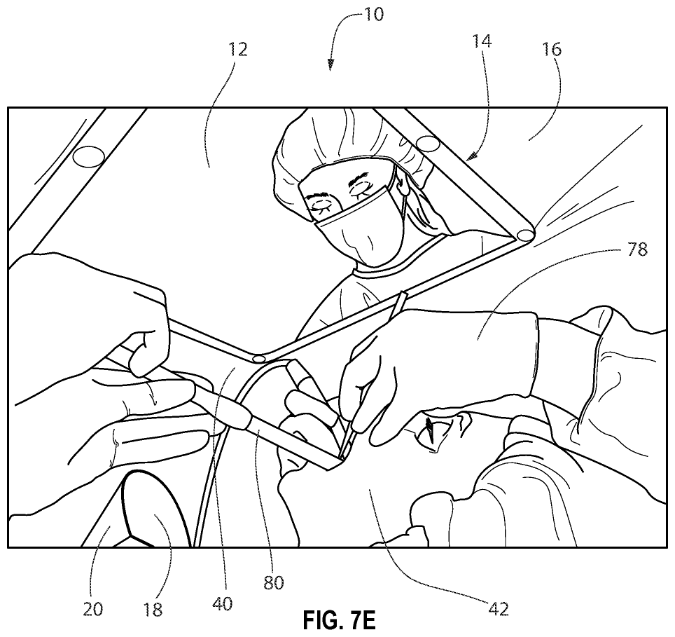

FIGS. 7A to 7E show various views of one optional embodiment of a system 10 that is used with a patient undergoing a dental procedure, although it should be understood that the system 10 may be adapted for use in other medical procedures or even non-medical procedures on people, such as cosmetic services. The system 10, in whichever context it is used, is particularly useful for preventing spread of aerosol (and airborne pathogens that may be present on or in such aerosol) during any of the aforementioned procedures.

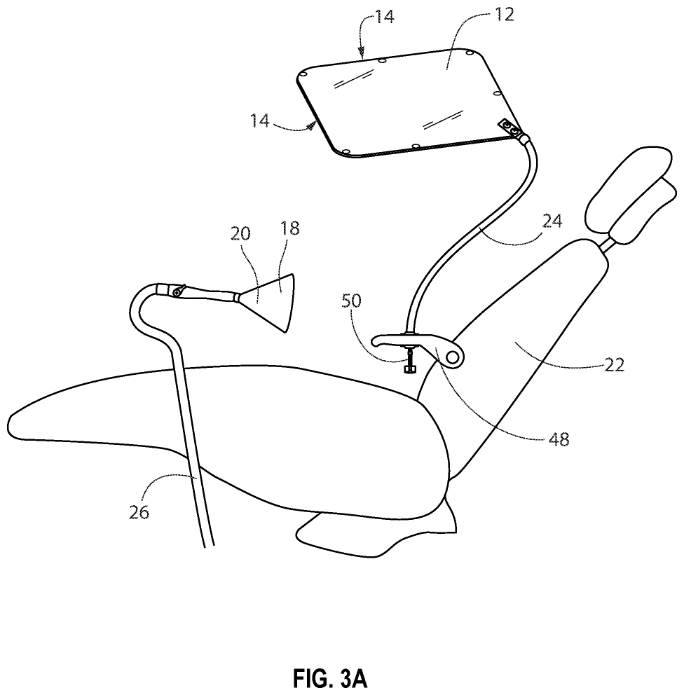

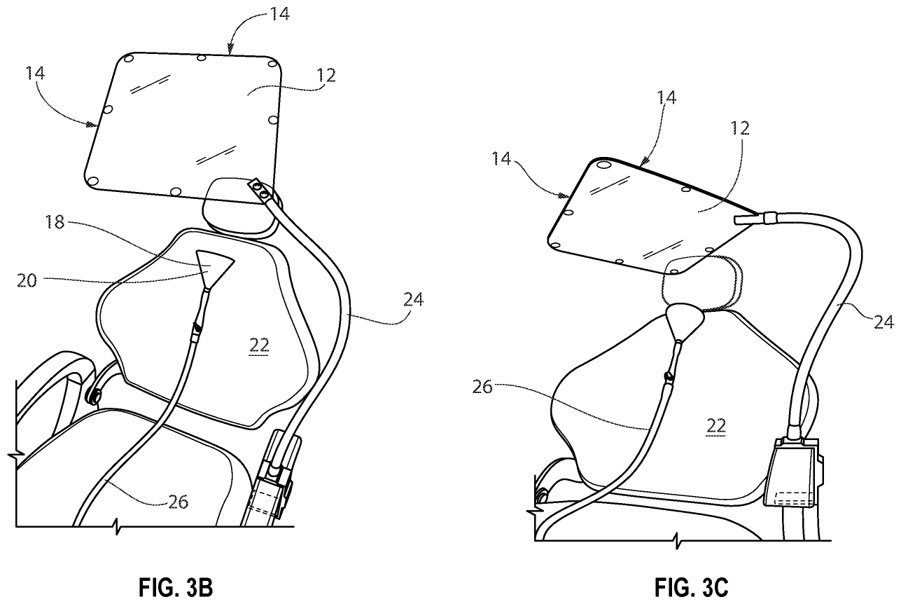

Various constituent components and subassemblies that make up embodiments of the system 10 are illustrated in FIGS. 1 to 6B. The system 10 (as fully assembled and implemented in FIGS. 7A to 7E) includes a containment unit 11 configured for positioning over a portion of a patient 42, preferably while the patient 42 is sitting or lying on a surface, e.g., a chair 22. For example, in the case of a dental procedure, the containment unit 11 may be positioned at least over the patient's head, neck and upper torso, e.g., as shown in FIGS. 7A to 7E, while the patient 42 is reclining on a chair 22 that is embodied in FIGS. 3A to 3C and 7A to 7E as a dental chair. It should be understood that various types of chairs or other patient support platforms may be utilized in conjunction with the system 10, depending on the particular application. Nonlimiting examples include dental chairs, operating tables and upright chairs for cosmetic applications, among others. When positioned over a patient 42 as shown in FIGS. 7A to 7E, the containment unit 11 defines a confined space 40 around (including over) the portion of the patient 42 that is covered and the surface supporting the patient (e.g., chair 22).

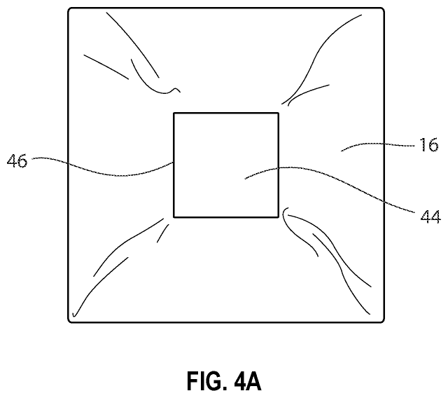

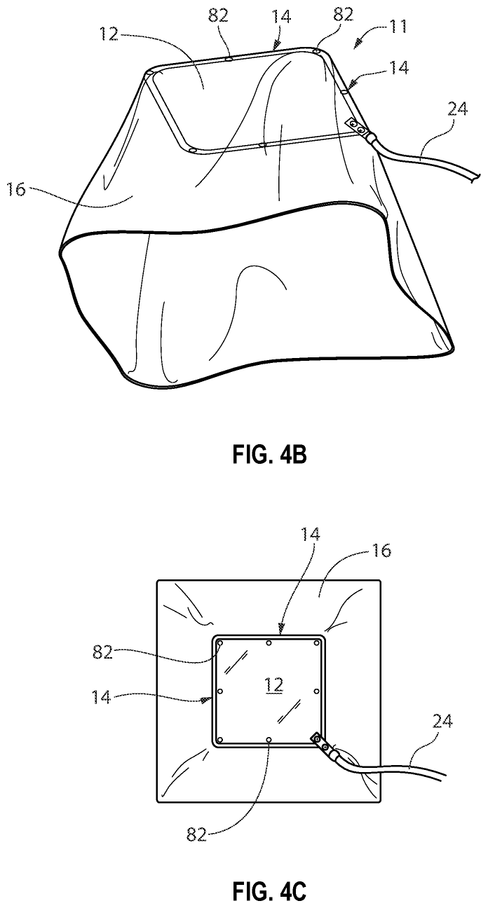

The containment unit 11 may include a rigid or semi-rigid transparent shield 12 that is bounded by an outer periphery 14. The containment unit 11 may further include a transparent or translucent flexible bib 16 extending from at least a majority of the outer periphery 14. As shown in FIGS. 4B and 4C, the bib 16 optionally extends from the entirety of (i.e., 100% of) the outer periphery 14. In alternative embodiments, the bib 16 extends from the majority of but less than the entirety of the outer periphery 14, e.g., at least 75% thereof, optionally at least 80% thereof, optionally at least 85% thereof, optionally at least 90% thereof, optionally at least 95% thereof. In addition, as shown in FIGS. 4B, 4C and 7A to 7E, the bib 16 extends downwardly from the outer periphery, essentially hanging under force of gravity. In alternative embodiments, at least a portion of the bib may extend in a non-downward direction from a shield. For example, where a patient is sitting upright and the shield is oriented along a substantially vertical plane (relative to the ground) between the patient and a professional administering a procedure to the patient, portions of the bib may extend horizontally from the outer periphery of the shield. In such embodiments, the bib is optionally removably secured to the patient and/or a structure adjacent the patient, e.g., the chair supporting the patient.

The bib 16 may be of any shape that fits a given shield configuration. For example, in the case of the substantially rectangular or square shaped shield 12 shown herein, an exemplary bib 16, as shown in FIG. 4A, is correspondingly rectangular or square shaped (when laid out flat) and optionally includes a central opening 44 defining an inner bib periphery 46 that is configured to fasten to and extend from a portion of the shield 12 adjacent the outer periphery 14 of the shield 12.

Accordingly, in any embodiment, the bib 16 may be secured, preferably removably secured, to the shield 12. In this way, a user may optionally clean and decontaminate the shield 12 for reuse and replace the bib 16 after each use. Various nonlimiting means for removably fastening the bib 16 to the shield are contemplated. For example, optionally, the bib 16 is secured to the shield 12 with an adhesive (e.g., adhesive tab) or a tacky putty material. The optional fasteners 82 depicted in the figures are adhesive tabs, to which the bib 16 removably adheres. Alternatively, the bib is secured to the shield with clips. Alternatively, the bib is secured to the shield with hook and eye or nub and eye type fasteners. Alternatively, the bib is secured to the shield with hook and loop (e.g., VELCRO® brand) fasteners. The type of fastener used to secure the bib 16 to the shield 12 is not particularly limited. The bib 16 is optionally disposable. The shield 12 may either be disposable or easily cleaned and decontaminated for reuse.

The system 10 may further include an inlet 18 configured to be provided within the confined space 40. The inlet 18 is preferably movable independently of the containment unit 11. The inlet 18, which is optionally in the form of a funnel 20, is configured to facilitate evacuation of air from the confined space 40. The evacuation may be, for example, suction evacuation. For example, the inlet 18 may be coupled to a lumen 26 (e.g., tubing, hosing, conduit, channel, passageway or the like) that is connected to a vacuum source to provide suction evacuation of air. In this way, when the system 10 is in use with a patient, aerosol generated by the patient is trapped within the containment unit 11 and directed towards the inlet 18, which, in turn, evacuates the aerosol from the containment unit 11, thereby preventing spread of the aerosol. Alternatively, the air may be evacuated through the inlet 18 using a fan, blower, negative pressure or other means to draw or convey air.

By virtue of the positioning and configuration of the shield 12 in relation to the patient 42 and the bib 16 extending from at least the majority of the outer periphery 14 of the shield 12 and thus at least substantially surrounding and enclosing the confined space 40, the confined space 40 is preferably substantially isolated from the air and environment outside the containment unit 11. This configuration facilitates creation of a negative pressure zone within the confined space 40 when the inlet 18 is positioned within the confined space 40 to evacuate air therefrom. An advantage of this localized negative pressure zone is that it enables removal of aerosol without the aerosol spreading throughout a room or building.

Optionally, one or more filtration elements may be provided in the inlet 18 or downstream from the inlet 18 to trap particulates or otherwise decontaminate air drawn into the inlet 18 from the confined space. Such filter elements may include particulate filters, such as one or more high-efficiency particular air (HEPA) filters and/or ultra-low particulate air (ULPA) filters. Alternatively, or in addition, one or more volatile organic compound filters, e.g., activated carbon filters, may be provided. Alternatively, or in addition, one or more germicidal filters, such as ultra-violet light sources, may be provided. The intended effect of such filters is to decontaminate the air (and aerosol contained therein) evacuated through the inlet 18, e.g., via suction evacuation, when the inlet is coupled to a lumen that is connected to a vacuum source.

The system 10 optionally comprises a mounting arm 24 that is coupled to the shield 12 to retain the containment unit 11 over a patient. Optionally, the mounting arm 24 is movable along at least one plane to facilitate and optionally adjust positioning of the containment unit 11. For example, the arm may have one or more bend joints and/or swivel joints to give the user flexibility to achieve precision in desired placement and positioning of the containment unit 11. In an optional aspect, the mounting arm may be gooseneck shaped or S-shaped. Optionally, FIGS. 1 and 3A to 3C show an exemplary mounting arm 24 made of a bendable material that retains its shape once manually configured to the desired shape, e.g., into a gooseneck. Optionally, in any embodiment in which a mounting arm is utilized, the mounting arm may be attached to a chair 22 (e.g., dental chair), a ceiling, a wall or a free-standing base (e.g., akin to a floor lamp).

In one exemplary embodiment shown in FIGS. 1, 3A, 6A and 6B, the mounting arm 24 may be secured to a chair 22, e.g., onto an armrest 48 (or another appendage) of the chair 22 with a clamp mechanism 50. The clamp mechanism 50 optionally comprises a U-shaped bracket 52 having a top portion 54, a bottom portion 56 and a side portion 58 bridging the top portion 54 to the bottom portion 56. Optionally the top portion 54 includes a top connection port 60 onto which the mounting arm 24 may be coupled, optionally threadedly coupled. Optionally, the side portion 58 includes a side connection port 62 onto which the mounting arm 24 may be coupled, optionally threadedly coupled. The two connection ports 60, 62 are optionally positioned 90° from each other, providing a user with flexibility in positioning of the mounting arm 24 and containment unit 11.

The clamp mechanism 50 further optionally includes a threaded shaft 64 comprising a knob 66 optionally at one end thereof and an engagement member 68 at the other end. The threaded shaft 64 protrudes through a hole 70 in the bottom portion 56 of the bracket 52 and engages complementary threads on the internal surface of the hole 70 such that manual rotation of the threaded shaft 64, e.g., using the knob 66, translates the threaded shaft 64 (and consequently the engagement member 68) in a direction along the central axis of the threaded shaft 64. In this manner, the gap between the engagement member 68 and the top portion 54 of the bracket 52 may be selectively widened or narrowed. A user may utilize the clamp mechanism 50, for example, by placing a chair armrest 48 in the gap between the engagement member 68 and the top portion 54 of the bracket 52 and tightening that gap so that the respective surfaces tightly clamp down onto and grip the armrest 48, thereby removably securing the clamp mechanism 50 and thus the mounting arm 24 to the armrest 48 (see FIG. 3A).

Both the shield 12 and bib 16 are preferably made from polymer. However, the nature and rigidity of the respective components and thus the selection of polymers, would likely differ as between the two components. It is important the shield 12 is transparent and the bib 16 is at least translucent, if not transparent, to enable a medical professional to sufficiently see through, to be able to get a clear view of the patient in order to perform the desired procedure on the patient. The specific polymers selected for the respective components are not limited. Exemplary polymers for the shield 12 include plexiglass (polymethylmethacrylate acrylic) polycarbonate, polyolefin, cyclic olefin polymer, cyclic olefin copolymer, glycol modified polyester, polystyrene and combinations of the foregoing. Optionally, the shield 12 is made from a non-porous material to facilitate easy cleaning and decontamination. In addition, the shield 12 should be impermeable or substantially impermeable to aerosols so as to inhibit their spread. Exemplary polymers for the bib 16 include polyethylene, polypropylene, nylon and combinations of the foregoing. Optionally, the shield 12 is treated or coated with one or more of an antifogging material, an anti-glare material or an antimicrobial material.

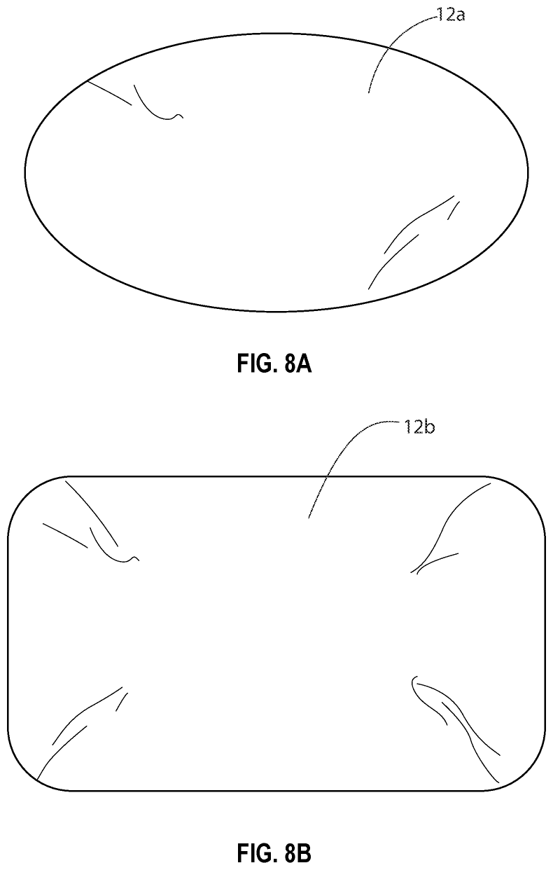

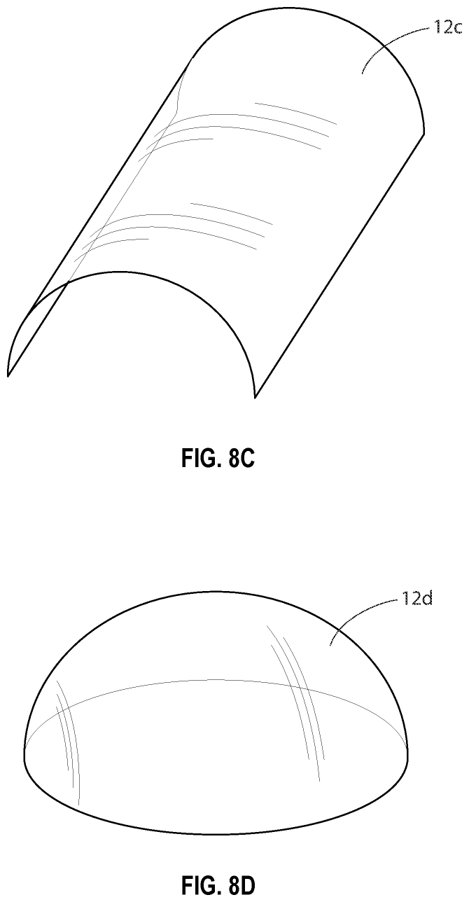

The shape of the shield 12 is not particularly limited. In the exemplary embodiment shown in FIGS. 1, 3A-3C, 4B, 4C, 5 and 7A to 7E, the shield 12 may be in the shape of a flat rectangular (e.g., square) member optionally having rounded corners. However, many alternative shapes and configurations of shields according to the disclosed concept are contemplated. The shield may effectively have a two-dimensional or three-dimensional surface. Non-limiting examples of alternative shield designs are shown in FIGS. 8A to 8D. The shield 12a of FIG. 8A is flat and oval in shape. The shield 12b of FIG. 8B is flat and rectangular in shape with rounded corners. The shield 12c of FIG. 8C is an elongate member having an arcuate cross section, i.e., shaped like the partial section of a tube cut longitudinally. The shield 12d of FIG. 8D is hemispherical in shape. Alternatively, the shield may be in the shape of an elongate hemi-ellipsoid, for example. These are all merely exemplary, as it should be understood that the shield shape and configuration may be adapted as needed for a given application.

The bib 16 may extend, e.g. downwardly, from an entire length of the outer periphery 14 of the shield 12 or from less than the entire length (albeit, still from a majority of the length) of the outer periphery 14. The purpose of the bib 16 is to extend the cover function of the shield 12 to form the containment unit 11, which traps aerosol generated during a cosmetic, medical or dental procedure, within the confined space 40. In serving this function, the bib 16 (and containment unit 11 generally) may help facilitate directional flow of aerosol towards the inlet 18 to evacuate the aerosol from the confined space 40. While the bib 16 may typically extend downwardly under force of gravity, depending on the positioning of the shield 12 in a given application, the bib may extend in other directions, e.g., horizontally.

The inlet 18 is not limited to any particular shape or configuration. In an optional embodiment, the inlet 18 is configured as a funnel 20. Optionally, as best seen in FIG. 2, the funnel 20 has a large opening 30 surrounded by an outer funnel periphery 36 and funnel inner surface 34 converging inward from the outer funnel periphery 36 to a small opening 32, which optionally leads to the lumen 26 to which the inlet 18 is coupled. A portion of the outer funnel periphery 36 and optionally a portion of an outer funnel surface 38 is flat or nearly flat. This configuration allows the funnel 20 to stably rest on its flat side, e.g., on the torso of a patient (see FIG. 7D), when in use.

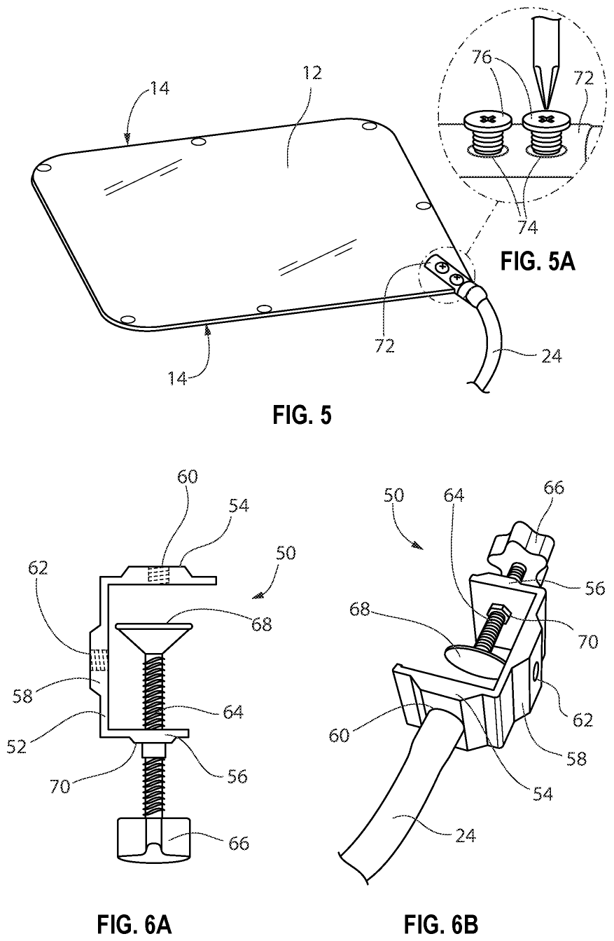

In an optional embodiment, the disclosed concept is directed to a chair 22 for supporting a patient undergoing a cosmetic, medical or dental procedure. The chair 22 is coupled to a portion of the system 10 for preventing spread of aerosol during the procedure described herein. In such an embodiment, the mounting arm 24 is secured to the chair 22, e.g., the armrest 44 of the chair 22. The mounting arm 24 is also to the shield 12 of the containment unit 11 to facilitate positioning of the containment unit 11 over the chair 22. The mounting arm 24 is movable along at least one plane to adjust positioning of the containment unit 11. Optionally, as best seen in FIGS. 5 and 5A, the mounting arm 24 comprises an extension 72 configured to be coupled to the shield 12. Optionally, the extension 72 is connected to the mounting arm 24 by a swivel joint, permitting at least some degree of axial rotation (if desired) of the extension 72, and thus the shield 12, relative to the mounting arm 24. This enables the user to slightly rotate the shield about the axis of the swivel joint, e.g., if initial positioning of the shield causes a glare from the user's vantage point.

Various means for coupling or fastening the extension 72 to the shield 12 are contemplated. In one non-limiting exemplary embodiment, the extension 72 comprises one or more screw holes 74 that receive corresponding screws 76 to fasten the shield 12 to the mounting arm 24. In an optional embodiment, the extension 72 is fastened to a corner of the shield 12, which Applicant has found, especially when using a rectangular or square shield, helps to facilitate a professional's reach and access to the patient positioned under the containment unit 11.

Optionally, the disclosed concept is also directed to methods for preventing spread of aerosol during a cosmetic, medical or dental procedure through use of the system 10 described herein. Optionally, this may be used for any procedure in which risk of patient-generated aerosol is a concern. “Patient-generated aerosol” may include any aerosol emanating from a patient that is self-generated (e.g., from the breath, a sneeze or a cough) or that is generated and/or scattered off of a patient's tissue by a cosmetic, medical or dental instrument (e.g., dental drill or air-water syringe used on a patient's mouth). For example, the system 10 may be used to cover the patient's entire face and neck and at least a portion of the patient's upper torso, so as to contain aerosol generated by the patient's mouth, optionally during a dental procedure or hospitalization of a highly contagious patient and/or patient particularly susceptible to airborne illnesses.

Where the method is for use in conjunction with a dental procedure, for example, it is possible that a dentist or dental hygienist would utilize suction in a patient's mouth to evacuate saliva generated during the procedure. However, the inlet 18 described herein is separate from that, is positioned differently and is used for another purpose. The inlet 18 is not positioned in a patient's mouth, but is rather placed elsewhere within the confined space 40, preferably towards a bottom end thereof, such as on the patient's chest or torso generally. The inlet 18, which may be coupled to a lumen 26 connected to a vacuum source provides evacuation, e.g., suction evacuation, of air from the confined space 40, thereby evacuating patient-generated aerosol, e.g., resulting from the cleaning or drilling of teeth. According to this method, the containment unit 11 traps the aerosol and facilitates directional flow of the aerosol towards the inlet 18. The inlet 18 helps create a negative pressure zone within the containment unit 11 and evacuates the aerosol from the containment unit 11. This safely removes the (potentially infectious) aerosol from the vicinity of the patient and substantially prevents such aerosol from spreading in the room (i.e., the ambient environment generally). In this way, the method thus prevents spread of the aerosol. Simultaneously, the system and methods described herein may also protect the patient from airborne pathogens outside of the containment unit 11.

Notably, e.g., as shown in FIGS. 7A to 7E, the patient's mouth (i.e., the relevant portion of the patient) is visible from outside of the containment unit 11 to enable a dental professional to see within the patient's mouth, to safely and effectively perform the procedure on the patient. Further, the flexible nature of the bib 16 enables the dental professional to readily manually reach into the containment unit 11 (e.g., by reaching underneath) to perform the procedure, while still allowing the bib 16 to substantially cover the patient and thus facilitate containment of the aerosol. It is noted that when a professional's arm reaches under the bib, small gaps around the arm may be formed. However, the containment unit 11 is still able to perform its function of substantially trapping patient-generated aerosol while the medical or dental professional is reaching therein to perform the procedure.

Accordingly, in any embodiment of the methods and systems described herein, the site of the patient's body undergoing a procedure is visible from outside of the containment unit to enable a professional who is performing the procedure to see the site so as to perform the procedure within the containment unit. For example, as shown in FIGS. 7A to 7E, where the site of the procedure is the patient's mouth or teeth (e.g., a dental procedure) and the professional can see inside the patient's mouth from outside the containment unit, the containment unit is positioned over the patient's entire face and neck and at least a portion of the patient's upper torso, so as to contain patient-generated aerosol. In this way, the containment unit is positioned and configured such that the professional can extend a hand 78 or an instrument 80 into the containment unit 11 by reaching underneath the bib 16 to perform the procedure.

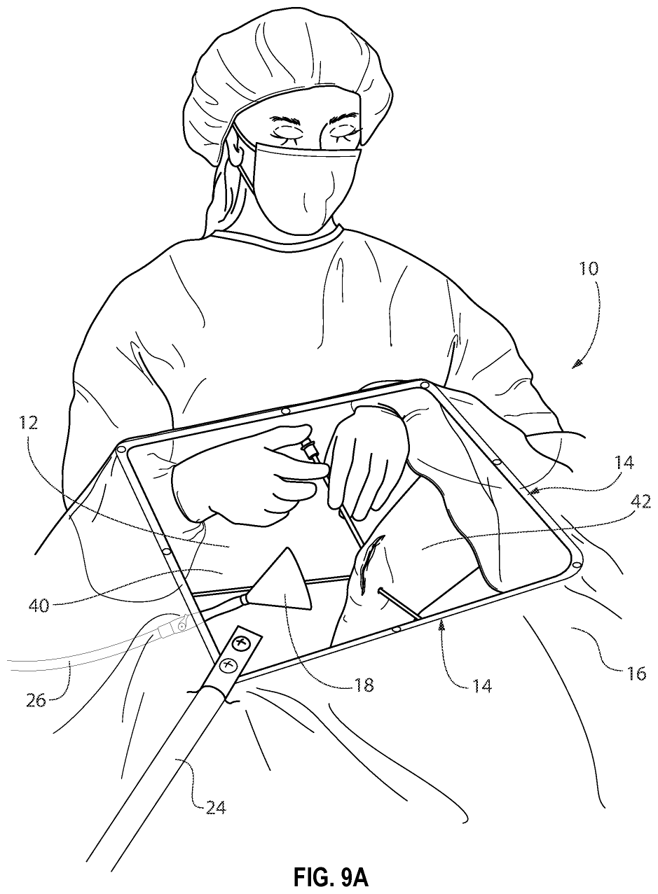

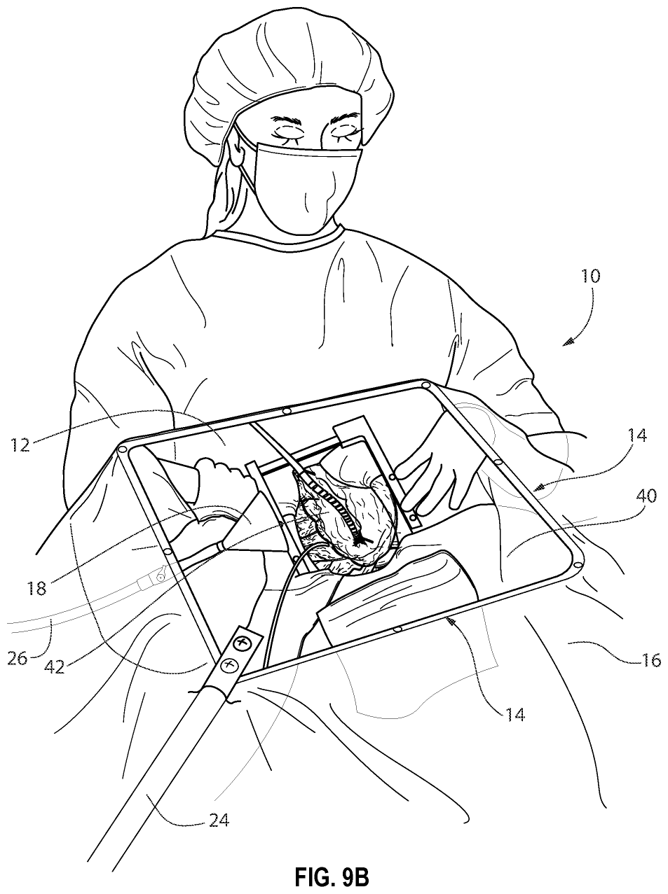

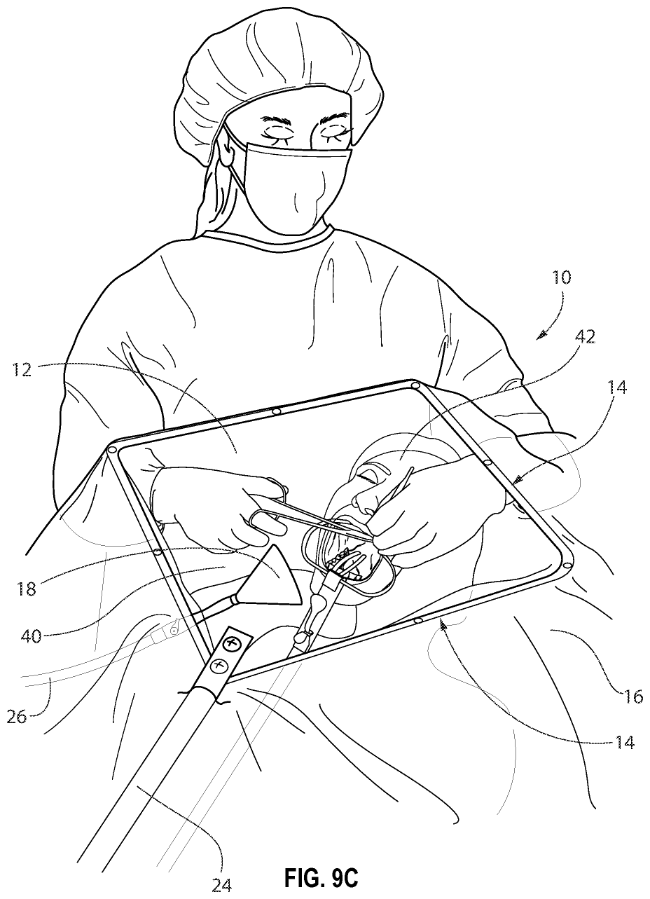

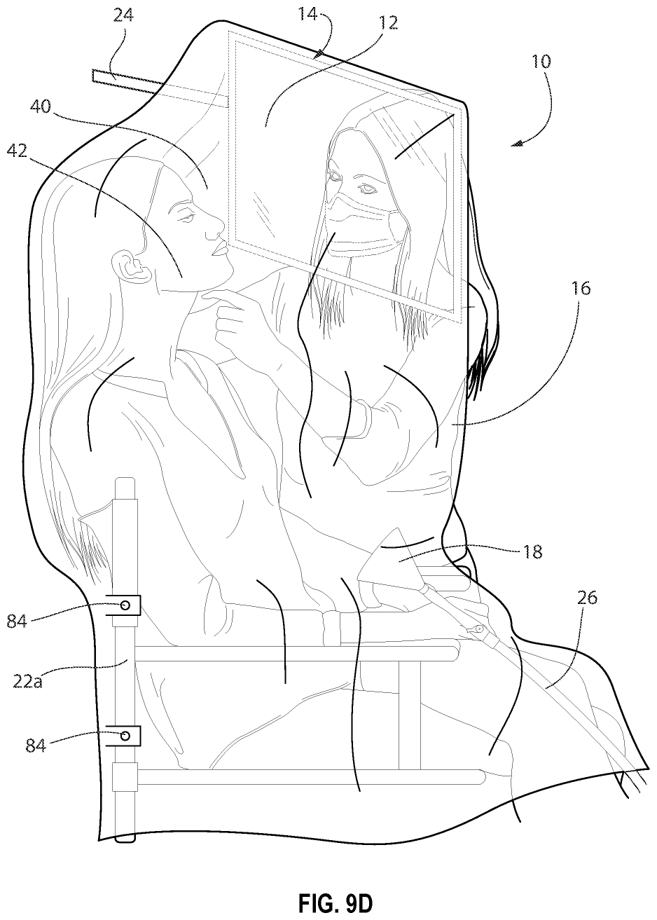

FIGS. 9A to 9D illustrate use of the systems and methods described herein in contexts aside from dentistry. For example, FIGS. 9A to 9C show use of the system 10 and associated methods in medical and surgical applications. In particular, the system 10 is being used in FIG. 9A on a patient undergoing knee surgery, in FIG. 9B on a patient undergoing open heart surgery and in FIG. 9C on a patient undergoing a tonsillectomy. These examples show that risks associated with airborne pathogens and patient-generated aerosol where the site of a given procedure is a patient's mouth, or not. For example, a bone saw used in an orthopedic surgery may generate aerosol, the spread of which may be addressed using systems and methods described herein.

FIG. 9D shows use of the system 10 and associated methods used with a “patient” or customer receiving facial cosmetic treatment from a beautician. Uniquely, in this illustration, the customer is sitting upright in a chair and the shield is oriented along a substantially vertical plane relative to the ground, between the patient and the beautician. Portions of the bib 16 in this configuration extend horizontally from the outer periphery 14 of the shield 12. The bib 16 may be removably secured to the customer 42 and/or the chair 22a, such as the back of the chair (or another structure adjacent the patient) using fasteners known in the field (e.g., adhesive, adhesive tab, putty, clip, hook and eye, nub and eye, hook and loop, straps, ties or static cling, for example). In the non-limiting example shown in FIG. 9D, the bib 16 is fastened to the back of the chair 22a with straps 84.

In another optional aspect, the systems and methods herein may be utilized in treatment of a patient having an infectious disease that is communicable through airborne transmission of pathogens from the patient's mouth (e.g., through the patient's breath, cough, sneeze and saliva). For example, in a hospital or home setting, the system may be placed over the patient's head, neck and a portion of his/her torso (e.g., like in the dental procedure shown in FIGS. 7A-7E) to trap and remove patient-generated aerosol containing contagious airborne pathogens. The system may be used in this way during a procedure on the patient or when no procedure is being administered (e.g., for a period of hours or days), in order to reduce the likelihood of spread of the patient's infection to others in the room or building in which the patient is housed. The system may create a localized negative pressure zone for the patient in a manner that keeps the patient comfortable and able to breathe easily, while protecting others. As such, the system may be used as a form of personalized protective equipment (PPE) to stop the spread of a pathogen, e.g., influenza, meningitis, or COVID-19, from a contagious patient, without placing a confining piece of PPE (e.g., mask or face shield) directly onto the nose and mouth of a patient. The combination of the shield and bib in this context enables healthcare providers to occasionally reach into the confined space to administer care or diagnostics to the patient, while reducing risk of infection to the provider(s) and others in the room or building.

In an optional aspect, the disclosed concept may be directed to the containment unit itself (without the inlet).

In an optional aspect, the disclosed concept may be directed to a kit that includes components of the system disclosed herein.

The systems, methods and components described herein are seriously needed to enable dentistry (and other procedures on patients) to be practiced while protecting people in the surrounding environment as well as the patient himself or herself from airborne pathogens.

While the invention has been described in detail and with reference to specific examples thereof, it will be apparent to one skilled in the art that various changes and modifications can be made therein without departing from the spirit and scope thereof.

What is claimed is:

1. A system for preventing spread of aerosol during a cosmetic, medical or dental procedure, the system comprising:
a. a containment unit configured for positioning over a portion of a patient so as to define a confined space around the portion of the patient, the containment unit comprising:
i. a rigid or semi-rigid transparent shield that is bounded by an outer periphery; and
ii. a transparent or translucent flexible bib extending from the outer periphery; and
b. an inlet configured to be provided within the confined space, wherein the inlet is configured to facilitate evacuation of air from the confined space.

2. The system of claim 1, further comprising a mounting arm that is coupled to the shield to facilitate positioning of the containment unit over the portion of the patient, wherein the mounting arm is movable along at least one plane to adjust positioning of the containment unit.

3. The system of claim 1, wherein the containment unit is configured to trap the aerosol within the confined space so as to facilitate directional flow of the aerosol towards the inlet, wherein the inlet is coupled to a lumen connected to a vacuum source to provide suction evacuation of air from the confined space.

4. (canceled)

5. The system of claim 1 wherein the shield is made from a polymer or glass that is treated or coated with an antifogging material.

6. The system of claim 1, wherein the bib extends from a majority of the outer periphery.

7. The system of claim 6, wherein the bib extends from at least 75% of the outer periphery.

8. The system of claim 6, wherein the inlet is coupled to a lumen connected to a vacuum source to provide suction evacuation of air from the confined space so as to create a negative pressure zone within the confined space.

9. The system of claim 1, wherein the inlet is a funnel with an outer funnel periphery, wherein the outer funnel periphery has a flat portion.

10. (canceled)

11. (canceled)

12. The system of claim 1, further comprising one or more filtration elements in the inlet or downstream from the inlet for trapping particulates or decontaminating the air drawn into the inlet from the confined space.

13. The system of claim 1, wherein the containment unit is positioned over the patient's entire face and neck and at least a portion of the patient's upper torso, so as to contain aerosol generated from the procedure, wherein the inlet is coupled to a lumen connected to a vacuum source to provide suction evacuation of air from the confined space, thereby evacuating the aerosol.

14. The system of claim 1, further comprising:
c. a mounting arm that is coupled to the shield to facilitate positioning of the containment unit over the portion of the patient, wherein the mounting arm is movable along at least one plane to adjust positioning of the containment unit; and
d. one or more filtration elements in the inlet or downstream from the inlet for trapping particulates or decontaminating the air drawn into the inlet from the confined space.

15. A chair for supporting a patient undergoing a cosmetic, medical or dental procedure, wherein the chair is coupled to a portion of the system of claim 2.

16. (canceled)

17. A method for preventing spread of aerosol during a cosmetic, medical or dental procedure, the method comprising:
a. providing a containment unit over a portion of a patient so as to define a confined space around the portion of the patient, the containment unit comprising:
i. a rigid or semi-rigid transparent shield that is bounded by an outer periphery; and
ii. a transparent or translucent flexible bib extending from the outer periphery; and
b. providing an inlet within the confined space to facilitate evacuation of air from the confined space.

18. The method of claim 17, further comprising providing a mounting arm that is coupled to the shield to facilitate positioning of the containment unit over the portion of the patient, the mounting arm being movable along at least one plane to adjust positioning of the containment unit.

19. The method of claim 17, wherein the bib extends from a majority of the outer periphery.

20. The method of claim 19, wherein the inlet is coupled to a lumen connected to a vacuum source to provide suction evacuation of air from the confined space so as to create a negative pressure zone within the confined space.

21. The method of claim 17, wherein the containment unit is positioned over the patient's entire face and neck and at least a portion of the patient's upper torso, so as to contain aerosol generated from the procedure which is performed on the patient's mouth.

22. The method of claim 21, wherein the containment unit traps the aerosol within the confined space so as to facilitate directional flow of the aerosol towards the inlet, wherein the inlet is coupled to a lumen connected to a vacuum source to provide suction evacuation of air from the confined space, thereby evacuating the aerosol.

23. The method of claim 17, wherein the aerosol is generated from the procedure and the site of the patient's body undergoing the procedure is visible from outside of the containment unit to enable a professional who is performing the procedure to see the site so as to perform the procedure within the containment unit.

24. The method of claim 23, wherein the site of the procedure is the patient's mouth or teeth and the professional can see inside of the patient's mouth from outside the containment unit, wherein the containment unit is positioned over the patient's entire face and neck and at least a portion of the patient's upper torso, so as to contain aerosol generated from the procedure.

25. The method of claim 24, wherein the containment unit is positioned and configured such that the professional can extend a hand or an instrument into the containment unit by reaching underneath the bib to perform the procedure.

26. The method of claim 17, wherein the inlet is a funnel with an outer funnel periphery and the outer funnel periphery has a flat portion.

27. (canceled)

28. A kit for preventing spread of aerosol during a cosmetic, medical or dental procedure comprising:
a. a rigid or semi-rigid transparent polymer or glass shield that is bounded by an outer periphery, wherein the outer periphery is configured to have a bib secured thereto;
b. a transparent or translucent flexible polymer bib configured to be secured to a majority of the outer periphery so as to form a containment unit configured to be placed over a portion of a patient so as to define a confined space around the portion of the patient;
c. an inlet configured to be placed within the confined space and coupled to a lumen connected to a vacuum source to provide suction evacuation of air from the confined space; and
d. a mounting arm configured to be coupled to the shield to facilitate positioning of the containment unit over the portion of the patient, wherein the mounting arm is movable along at least one plane to adjust positioning of the containment unit.
